(19) United States
(12) Patent Application Publication
(10) Pub. No.: US 2025/0161116 A1
(43) Pub. Date: May 22, 2025

(54) WOUND DRESSING

(71) Applicant: CINOGY GMBH, Duderstadt (DE)

(72) Inventors: Dirk WANDKE, Heilbad Heiligenstadt (DE); Benedikt BUSSE, Overath (DE); Jan-Hendrik HELLMOLD, Duderstadt (DE)

(21) Appl. No.: 18/841,864

(22) PCT Filed: Mar. 3, 2023
(86) PCT No.: PCT/EP2023/055464

§ 371 Date: Aug. 27, 2024

(30) Foreign Application Priority Data
Mar. 4, 2022 (DE) 10 2022 105 186.2

(51) Int. Cl. A61F 13/00; A61F 13/02; A61F 13/0206

(52) U.S. Cl. CPC A61F 13/00051; A61F 13/0209; A61F 13/0243; A61F 2013/00089

(57) ABSTRACT

The invention relates to a wound dressing for placement on a wound located on a skin surface with an electrode arrangement for forming a dielectric barrier plasma discharge by way of at least one electrode that can be supplied with a high AC voltage and is completely embedded in a flat dielectric, wherein the dielectric forms a wound-side application side and the electrode has intermediate spaces dispersed across its surface that are aligned with passage openings of the dielectric, the passage openings of the dielectric having smaller dimensions than the intermediate spaces of the electrode, so that the dielectric also completely covers the electrode in the area of the passage openings and extends from the wound-side application side to a rear side of the dielectric opposite the wound-side application side and is permeable to a fluid, characterized in that a fluid absorption layer for absorbing a fluid conducted through the passage openings of the dielectric from the wound-side application side to the rear side, which has a super-absorber or consists of such a super-absorber, rests against the opposite rear side of the dielectric.

The invention relates to a wound dressing for placement on a wound located on a skin surface.

To treat a wound on a skin surface, it is known to apply a wound dressing to the wound and the skin surface on which the wound is located to prevent the penetration of foreign bodies into the wound and to allow the absorption of fluids such as blood and/or wound exudate. Such a wound dressing is usually multi-layered and generally features an absorbent material, such as cellulose or cotton, and is provided to absorb and store blood and/or wound exudate.

DE 20 2004 009 429 U1 discloses an absorbent hygienic article with an absorbent core, which is composed of an absorbent fiber fleece and is covered on at least one side by a non-permeable film or a fleece web. Super-absorbent polymers may be added to the fiber material of the core.

Such super-absorbers are materials that are able to absorb a large quantity of liquids, such as blood and/or wound exudate, by means of a chemical-physical bond in particular. The absorption capacity of such super-absorbers is many times its own weight, wherein the materials generally swell and thus store the absorbed liquids and do not release them again, even under pressure. By using such super-absorbers as materials for the absorbent layer of a wound dressing, large quantities of blood and/or wound exudate in relation to the size and weight of the wound dressing can be absorbed, so that optimal wound treatment can be ensured for as long as possible.

It is also known for the wound dressing to comprise a self-adhesive layer on the wound side of the wound dressing, as is described, for example, in EP 2 338 449 A1. This ensures that the wound dressing remains in place over the wound to be treated, even over a longer period of time.

DE 10 2017 100 192 A1 describes a permanent wound dressing where the wound secretion can be extracted by means of a suction pump.

Furthermore, plasma treatment has been shown to be potentially beneficial for treating a wound. An electrode arrangement forming a wound dressing consisting of a dielectric and an electrode embedded in the dielectric is used to generate a dielectric barrier plasma discharge between a flat surface of the electrode arrangement and a surface to be treated, which serves as a counter electrode, by supplying the electrode of the electrode arrangement with a high AC voltage.

Such an electrode arrangement for a dielectric barrier plasma discharge is known, for example, from DE 10 2014 013 716 A1. The electrode arrangement disclosed therein has passage openings in the dielectric which are aligned with passage openings in the flat electrode, whereby the passage openings of the electrode are larger than the passage openings of the dielectric, so that the dielectric also completely covers the electrode in the area of the passage openings. The lower side of the dielectric facing towards the wound may be covered with a layer, e.g. of a gauze cellulose material or a material that preserves or encourages healing.

A similar electrode arrangement is also known from DE 10 2016 118 569 A1, in which the electrode arrangement is formed of at least two partial electrodes, which are insulated against each other and embedded next to each other in the dielectric. Neighbouring partial electrodes are supplied by a control device with compensating partial alternating voltages of opposite waveforms and voltage levels.

It is the task of the present invention to present an improved wound dressing with which the wound located on the skin surface can be treated with a dielectric barrier plasma discharge.

According to the invention, the task is solved with the wound dressing in accordance with claim 1. Advantageous embodiments of the invention are to be found in the corresponding sub-claims.

According to claim 1, a wound dressing for placement of a wound located on a skin surface is proposed, wherein the wound dressing comprises an electrode arrangement for forming a dielectric barrier plasma discharge by way of at least one electrode that can be supplied with a high AC voltage and is completely embedded in a flat dielectric.

The dielectric forms a wound-side application side facing the skin surface to be treated. The electrode has intermediate spaces distributed over its surface, which are aligned with passage openings of the dielectric, the passage openings of the dielectric having smaller dimensions than the intermediate spaces of the electrode, so that the dielectric also completely covers the electrode in the area of the passage openings and extends from the wound-side application side to a rear side of the dielectric opposite the wound-side application side and is permeable to a fluid.

According to the invention, it is now provided that a fluid absorption layer for absorbing a fluid conducted through the passage openings of the dielectric from the wound-side application side to the rear side, which has a super-absorber or consists of such a super-absorber, rests against the opposite rear side of the dielectric. The passage openings in the dielectric are designed in such a way that a fluid forming on the wound-side application side, such as wound exudate and/or blood, is channelled through the passage openings of the dielectric to the fluid receiving layer on the back of the electrode arrangement, so that the fluid can be absorbed and stored by the super-absorber. The cross-sectional size (diameter) of the passage openings can be designed in such a way that the absorption of the fluid by the fluid receiving layer is supported by a capillary effect in the passage openings.

This makes it possible to use an electrode arrangement for to heal a wound, as described for example in DE 10 2014 013 716 A1, including for wounds with a strong secretion of wound exudate, since any wound exudate that forms is drained off by the electrode arrangement to the super-absorber above it. Furthermore, the wound dressing can remain on the skin surface to be treated for an extended period because the fluid absorption layer with the super-absorber can absorb its own weight in fluid many times over.

The super-absorber is designed in such a way that the water absorption capacity of the super-absorber corresponds to the super-absorber's own weight many times over. As such, the super-absorber has a water absorption capacity that corresponds to at least three times its own weight, preferably at least five times its own weight.

The electrode is also completely shielded in the area of the passage openings of the dielectric, so that a wound exudate that has formed on the wound can be safely discharged to the super-absorber. Advantageously, the intermediate spaces in the electrode, which are at least partially occupied by the dielectric so as to form the passage openings, result in the electrode being securely held within the dielectric, which is especially advantageous if the electrode and the dielectric are designed to be flexible and thus have to adhere to various surface shapes of the skin surface to be treated.

The electrode can be designed to be flat, wherein the intermediate spaces for the passage openings in the dielectric are formed by recesses in the flat electrode. However, the electrode may also be designed to be wire-shaped or belt-shaped and be embedded in the dielectric in a snail-like or meandering shape. Intermediate spaces are formed between the wire-or belt-shaped electrode, said spaces being aligned with the passage openings in the dielectric.

The electrode arrangement, or more specifically the electrode, can be connected to or be a high-voltage generator in order to supply the electrode with a high AC voltage. The high-voltage generator can form part of a control device that controls the application of the high AC voltage on the electrode by means of the high-voltage generator. The high-voltage generator and/or the control device can be arranged outside of the wound dressing or outside of the electrode arrangement.

In this embodiment, the electrode arrangement may comprise a connecting piece for connecting the at least one electrode of the electrode arrangement to the high-voltage stage, especially as needed. In this case, an electrical supply line is guided from the electrode into the connecting piece, wherein the high-voltage stage can then be electrically contacted to the supply line of the connecting piece, e.g. by means of an electrical connection arrangement.

The connecting piece can be designed as a single piece with the dielectric and, in particular, made from the dielectric material. The connecting piece insulates the supply line of the respective electrode or partial electrode against the surroundings. A connection arrangement is provided at one of the ends of the supply line opposite the electrode in order to connect the supply line in the connecting piece to the high-voltage generator. In a simple embodiment, a recess is provided in the dielectric for this purpose, which exposes the end of the supply line to be contacted, thereby enabling contact with the high-voltage generator. The supply line is contacted within the insulation, wherein the connection point is outwardly insulated by the connection arrangement so as to ensure safe operation.

The connecting piece thus contains at least one electrical conductor per electrode or per partial electrode to apply an electric AC voltage to the respective electrode or partial electrode.

However, it is also conceivable that the high-voltage generator and, where applicable, the control device are integral components of the electrode arrangement in the form of a contact piece. Here, the high-voltage generator and, where applicable, the control device are integrated into the dielectric, for example, wherein the high-voltage generator is then electrically contacted to the electrode via an electrical connection extending in the dielectric.

In this case, no separate connecting piece is required to connect the electrode to a high-voltage stage of a high-voltage generator. Rather, all components required for generating a dielectric barrier plasma discharge (especially a high-voltage stage of the high-voltage generator as well as a control device and a self-supporting energy source, where applicable) are embedded into the contact piece, thus enabling self-supporting operation of the dielectric barrier plasma generation during treatment of the wound with the wound dressing. It can be provided for that the self-supporting electrical energy source can be recharged by an external energy source and that an adapter, especially a standardized adapter (such as a USB-C connector), is provided on the connecting piece to serve this purpose.

Ultimately, it is also conceivable to combine both options to achieve to greatest possible flexibility.

The electrode of the electrode arrangement can preferably be formed of multiple partial electrodes embedded in the dielectric that are insulated against each other, so that each of these partial electrodes can be supplied with a partial high AV voltage separately from each of the others. Preferably, the partial electrodes are at the same distance from an application surface of the dielectric facing towards the skin surface.

For practical purposes, the peak voltage of the high AC voltage(s) used can be between ±1 kV and ±100 kV. The alternating frequencies of the high AC voltages are, for practical purposes, between some 100 Hz and approximately 100 MHz. In this way, the pulse frequency, i.e. the frequency at which the individual pulses are emitted one after the other, can be more than 90 Hz, while the impulse frequency itself is more than 1 MHz.

According to one embodiment, it is provided for that the electrode arrangement is configured between the electrode that can be supplied with a high AC voltage and the skin surface of an electrically conductive body, which serves as a ground electrode, in order to form a dielectric barrier plasma discharge.

In this case, the skin surface with the wound to be treated is part of an electrically conductive body, which forms the ground electrode in the context of generating the dielectric barrier plasma discharge.

However, it is also conceivable that the electrode arrangement comprises a counter-electrode insulated against the electrode (the former also being embedded into the dielectric, for example) in order to achieve a dielectric barrier plasma generation between the electrode and the counter-electrode of the electrode arrangement when the electrode is supplied with a high AC voltage and the counter-electrode lies, for example, on a ground potential.

According to one embodiment, it is provided for that the electrode arrangement with the electrode and the dielectric are flexible. This makes it possible for the wound dressing, which is flexible overall, to adapt to any shape of skin surface. This achieves more intensive and improved plasma generation.

According to one embodiment, it is provided for that a wound contact layer rests against the wound-side application side of the dielectric, said layer being permeable to the fluid to be absorbed by the fluid absorption layer in the direction of the electrode arrangement. The wound contact layer can be made, for example, from a cotton material and renders the wound dressing comfortable to wear, even for longer periods of time. The wound contact layer may rest directly on the wound or skin surface, wherein the dielectric then rests directly on the rear side of the wound contact layer (the side facing away from the wound).

According to one embodiment, it is provided for that the electrode of the electrode arrangement has at least two partial electrodes that are arranged next to each other and insulated against each other by the dielectric, and that neighbouring partial electrodes are supplied by a control device with compensating partial alternating voltages of opposite waveforms and voltage levels. In this case, it can also preferably be provided for that the skin surface of the electrically conductive body serves as a ground electrode.

In the flat electrode arrangement, the partial electrodes are arranged next to one another in a plane and are at the same distance from an application surface of the dielectric facing towards the skin surface and are insulated against each other by the dielectric. The compensating partial alternating voltages of opposite waveforms and voltage levels results in an average potential that does not change over the period of high AC voltage, said potential corresponding to the ground potential of the ground electrode, meaning that accumulations of charge carriers on the patient's skin are prevented or reduced. Furthermore, a lower high-voltage can be used.

According to one embodiment, it is provided for that the partial electrodes are the same size.

According to one embodiment, it is provided for that the passage openings of the dielectric have a diameter of between 0.5 mm and 7 mm, preferably a diameter of between 0.75 mm and 5 mm.

According to one embodiment, it is provided that the area of all passage openings of the dielectric corresponds to an area of 0.1% to 60% of the electrode area enclosed by the outer edge of the electrode. Preferably, the area corresponds to 1% to 35% of the enclosed electrode surface.

According to one embodiment, it is provided for that the wound dressing has a wound-side outer layer and an opposite rear-side outer layer, which is facing away in relation to the skin surface, the fluid absorption layer being arranged between the wound-side outer layer and the rear-side outer layer, wherein an accommodation space is formed between the wound-side outer layer and the fluid absorption layer in which the electrode arrangement is arranged or can be inserted and wherein the wound-side outer layer is permeable to the fluid to be absorbed by the fluid absorption layer in the direction of the electrode arrangement.

In this embodiment, the wound-side outer layer, which as the wound contact layer comes into contact with the skin surface to be treated and the wound, is connected to the rear side outer layer on at least one side, preferably on two opposite sides, so that an accommodation space for the electrode arrangement is formed between the fluid absorption layer enclosed within and the wound-side outer layer. The electrode arrangement can be inserted into the accommodation space, e.g. as needed, and removed from the accommodation space following treatment or after the wound dressing has been removed from the patient and reused following suitable cleaning.

If the electrode arrangement is inserted into the accommodation space, the electrode arrangement rests with the wound-side application side against the inner wall of the wound-side outer layer on one side and with the opposite rear side of the dielectric against the fluid absorption layer with the super-absorber on the other. The fluid to be absorbed is then guided through the wound-side outer layer and through the passage openings of the dielectric of the electrode arrangement to the fluid absorption layer with the super-absorber.

It is likewise conceivable that the wound-side outer layer and the rear-side outer layer are connected to each other on three sides, thereby forming a pocket-like accommodation space.

It is likewise conceivable that the wound-side outer layer and the rear-side outer layer are connected to each other on all sides, so that the electrode arrangement is enclosed in the accommodation space. In this embodiment, only the electrical adapters potentially required are led out. Here, the electrode arrangement is enclosed in the accommodation space. It can be provided for that a resealable element is provided on one side, for example a zip, for opening the wound dressing if necessary.

According to one embodiment, it is provided for that a connecting piece of the electrode arrangement protrudes from the accommodation space of the wound dressing in order to connect the electrode to a high-voltage generator for supplying high AC voltage. This may refer to a connecting piece as previously described above.

According to one embodiment, it is provided for that the rear-side outer layer in particular is impermeable to liquids. In particular, the rear-side outer layer may not only be impermeable to liquids, but also breathable and impermeable to germs.

According to one embodiment, it is provided for that there are more than 20 passage openings of the dielectric, preferably more than 50, preferably more than 80, preferably more than 100 or preferably more than 125.

It is advantageous for the passage openings in the dielectric to be designed in such a way that the fluid transport is supported by a capillary effect in the direction of the super-absorber. In this case, the passage openings in the dielectric are so big that, in relation to the thickness of the dielectric, the capillary effect has such a positive effect that the fluid to be discharged is transported to the super-absorber.

The electrode arrangement can be produced in a molding process, in particular an injection molding process. The dielectric refers, in particular, to a moldable material.

The invention is explained in more detail by means of the attached figures. They show:

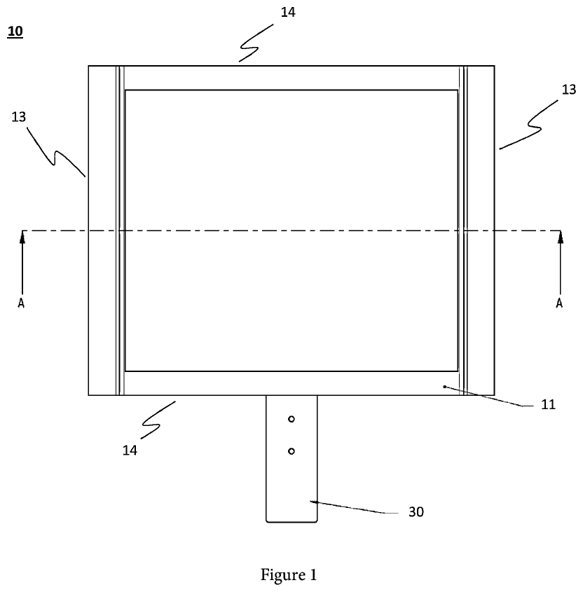

FIG. 1 general representation of the wound dressing according to the invention;

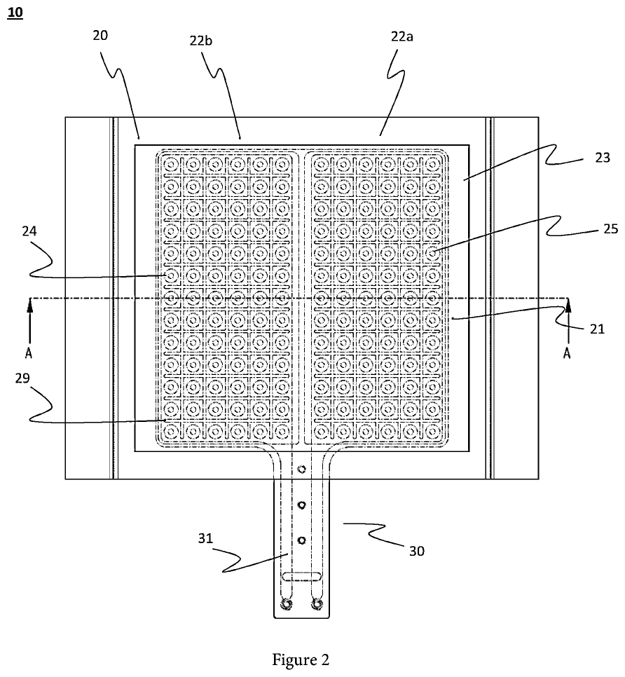

FIG. 2 representation of the wound dressing with a view of the electrode arrangement;

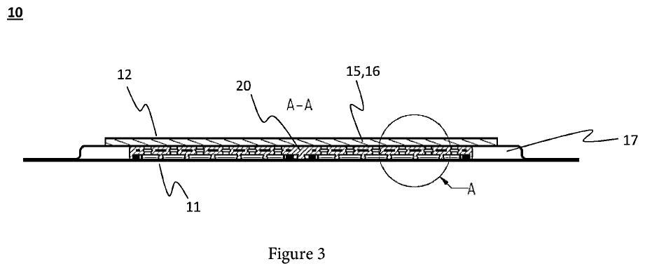

FIG. 3 sectional representation of figure 1/2;

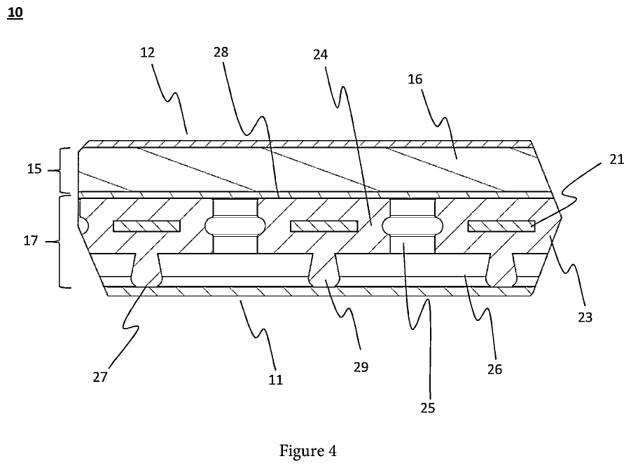

FIG. 4 enlargement of the section representation from section A of FIG. 3.

FIGS. 1 to 4 depict one embodiment of a wound dressing 10 in various representations and perspectives. FIGS. 1 and 2 comprise a view of the wound dressing 10 from the wound-side outer layer 11, while the rear-side outer layer 12 is covered. In the embodiment shown, the wound dressing 10 according to the invention has a rectangular base area, wherein the wound-side outer layer 11 is firmly connected to the rear-side outer layer 12 in an edge section 13. In an opening section 14, however, the wound-side outer layer 11 is not firmly connected to the reverse outer side 12, so that an opening can be formed for an intervention in the wound dressing 10.

The wound-side outer layer 11 can be a perforated PE film, for example. In particular, the wound-side outer layer 11 is designed in this case in such a way that a fluid, such as a wound exudate, which can form between the wound dressing 10 and the wound to be treated, reaches the interior of the wound dressing. In particular, the rear-side outer layer 12 can be designed to be impermeable to liquid. A fluid absorption layer 15 comprising a super-absorber 16 is located between the wound-side outer layer 11 and the rear-side outer layer 12.

An accommodation space 17, into which an electrode arrangement 20 is inserted, is formed between the fluid absorption layer 15 with the super-absorber 16 and the wound-side outer layer 11. The opening section 14 allows interventions in the accommodation space 17, so that the electrode arrangement 20 can be inserted into the accommodation space 17. Upon completion of the plasma treatment, the electrode arrangement 20 can be removed again, cleaned and reused, while the remaining components of the wound dressing 10 can be appropriately disposed of.

In the embodiment example in FIGS. 1 to 4, the electrode arrangement 20 has an electrode 21 with two partial electrodes 22a, 22b, which are firmly embedded in a dielectric 23.

The partial electrodes 22a, 22b are designed to be flat and have recesses or openings 24, which are intended to align with passage openings 25 in the dielectric 23. In this case, the passage openings 25 in the dielectric are smaller than the recesses 24 in the partial electrodes 22a, 22b, so that the electrodes 22a, 22b are also completely shielded in the area of the passage openings 25 in the dielectric.

If such an electrode arrangement 20 is now inserted into the accommodation space 17 formed in the wound dressing 10, the passage openings 25 in the dielectric form a fluid channel from the wound-side outer layer 11 to the fluid absorption layer 15 with the super-absorber 16. The electrode arrangement 20 then rests with an application side 27 of the dielectric 23 facing the wound-side outer layer 11 against the inner wall of the wound-side outer layer 11 on one side and with a rear side 28 of the dielectric 23 facing away against the fluid absorption layer 15 with the super-absorber 16 on the other, so that any wound exudate and/or blood forming on the wound-side outer layer 11 can then be guided through the electrode arrangement 20 to the super-absorber 16.

On the application side 27 of the dielectric 23 there is a spacer grid 29 formed by the dielectric 23, which has thin webs running longitudinally and transversely to it. The spacer grid 29 results in the formation of pre-chambers 26 for some or all passage openings 25, in which the dielectric barrier plasma can form and spread when the electrode 21 is supplied with a high AC voltage. If a wound exudate and/or blood forms in the wound area, the pre-chambers 26 can be individually filled and the wound exudate and/or blood drained off in the direction of the super-absorber by exploiting the capillary effect in the passage openings 25.

In order to create the electrode arrangement, a first dielectric layer can initially be formed by a pourable material in a mold into which the electrode 21 is placed. The electrode is then shielded on all sides by the dielectric by applying a second dielectric layer made of the pourable material. In the process, cores are inserted to form the passage openings 25, said cores being removed once the electrode arrangement has been created.

The dielectric 23 forms a connecting piece 30 on one side, into which a respective electrical supply line 31 extends separately for each partial electrode 22a, 22b. The connecting piece 30 protrudes beyond the outer edge of the wound dressing 10, especially in the area of the opening section 14, thereby enabling electrical contact of the electrode 21 to a high AC generator. A recess in the insulation is provided in an end section of the supply lines 31 so as to render the supply lines 31 accessible from the outside. A suitable connection arrangement then allows an electrical connection to the supply line 31 to be established in that the connection arrangement engages in the recess in the insulation and contacts the supply line within the insulation, wherein the connection point is outwardly insulated by the connection arrangement.

The rear-side outer layer 12 is designed in such a way that the fluid absorption layer 15 with the super-absorber 16, which is pressed into a powder form before the fluid is absorbed, is held in its shape.

### REFERENCE LIST

10 wound dressing
11 wound-side outer side
12 rear-side outer side
13 edge section
14 opening section
15 fluid absorption layer
16 super-absorber
17 accommodation space
20 electrode arrangement
21 electrode
22a,22b partial electrodes
23 dielectric
24 recesses/intermediate spaces in the electrode
25 passage openings in the dielectric
26 pre-chambers
27 contact side of the dielectric
28 rear-side of the dielectric
29 spacer grid
30 connecting piece
31 supply line in the connecting piece

1. A wound dressing for placement on a wound located on a skin surface, comprising:
an electrode arrangement for forming a dielectric barrier plasma discharge by way of at least one electrode supplied with a high AC voltage,
wherein the electrode arrangement is completely embedded in a flat dielectric comprising a wound-side application side,
wherein the at least one electrode has intermediate spaces dispersed across a surface of the at least one electrode that are aligned with passage openings of the flat dielectric,
wherein the passage openings have smaller dimensions than the intermediate spaces of the at least one electrode,
wherein the flat dielectric completely covers the at least one electrode in an area of the flat dielectric comprising the passage openings and extends from the wound-side application side to a rear side of the flat dielectric,
wherein the rear side of the flat dielectric is opposite the wound-side application side and is permeable to a fluid; and
a fluid absorption layer for absorbing a fluid conducted through the passage openings of the flat dielectric from the wound-side application side to the rear side, wherein the fluid absorption layer comprises or a super-absorber, wherein the fluid absorption layer rests against the rear-side of the dielectric.

2. The wound dressing according to claim 1, wherein a the cross-sectional size of the passage openings are designed such that absorption of fluid by the fluid absorption layer is supported by a capillary effect in the passage openings.

3. The wound dressing according to claim 1 further comprising a wound contact layer that rests against the wound-side application side of the flat dielectric, wherein the wound contact layer is designed to be permeable to fluid to be absorbed by the fluid absorption layer in a direction of the electrode arrangement.

4. The wound dressing according to claim 1 wherein the passage openings of the flat dielectric have a diameter of between 0.5 mm and 7 mm.

5. The wound dressing according to claim 1 wherein the area of the passage openings of the flat dielectric corresponds to an area of 0.1% to 60% of an electrode area enclosed by an outer edge of the at least one electrode.

6. The wound dressing (10) according to claim 1 further comprising a wound-side outer layer and an opposite rear-side outer layer that faces away in relation to a skin surface,
wherein the fluid absorption layer is arranged between the wound-side outer layer and the rear-side outer layer,
wherein an accommodation space is formed between the wound-side outer layer and the fluid absorption layer, wherein the accommodation space is configured for having the electrode arrangement arranged therein or inserted therein, and
wherein the wound-side outer layer is permeable to fluid to be absorbed by the fluid absorption layer in a direction of the electrode arrangement.

7. The wound dressing according to claim 6, further comprising a connecting piece of the electrode arrangement that protrudes from the accommodation space of the wound dressing configured for connecting the at least one electrode to a high-voltage generator for supplying a high AC voltage.

8. The wound dressing according to claim 1 wherein the passage openings of the flat dielectric comprise more than 20 passage openings.

9. The wound dressing according to claim 1 wherein a water absorption capacity of the super-absorber corresponds to the super-absorber's own weight many times over.

10. The wound dressing according to claim 1 wherein the electrode arrangement is configured to form a dielectric barrier plasma discharge.

11. The wound dressing according to claim 1 wherein the electrode arrangement is designed to be flexible.

12. The wound dressing according to claim 1 wherein the at least one electrode of the electrode arrangement comprises at least two partial electrodes that are arranged next to each other and insulated against each other by the flat dielectric, wherein the at least two partial electrodes comprises neighbouring partial electrodes that are supplied by a control device with compensating partial alternating voltages of opposite waveforms and voltage levels.

13. The wound dressing according to claim 12 wherein the partial electrodes are the same size.

14. The wound dressing (10) according to claim 6 wherein the wound-side outer layer and the rear-side outer layer are connected to each other on three sides, thereby forming a pocket-like accommodation space.
